(19) United States
(12) Patent Application Publication
(10) Pub. No.: US 2016/0051158 A1
(43) Pub. Date: Feb. 25, 2016

(54) HARMONIC TEMPLATE CLASSIFIER

(71) Applicant: Apple Inc., Cupertino, CA (US)

(72) Inventor: James M. SILVA, San Jose, CA (US)

(21) Appl. No.: 14/550,820

(22) Filed: Nov. 21, 2014

Related U.S. Application Data
(60) Provisional application No. 62/040,967, filed on Aug. 22, 2014.

(51) Int. Cl. A61B 5/024; A61B 5/00

(52) U.S. Cl. CPC A61B 5/02416; A61B 5/721; A61B 5/7246

(57) ABSTRACT

An algorithm for removing motion artifacts from a PPG signal in the frequency domain and a harmonic template classifier (HTC) algorithm and unit to determine heart rate are disclosed. In some examples, PPG signals can be processed in combination with accelerometer signals to remove unwanted artifacts in the frequency domain. For example, an acceleration mask can be generated and used to filter out acceleration contributions represented in the PPG signal. Additionally or alternatively, in some examples, an HTC unit can be configured to generate a heart rate correlation curve based on the correlation between frequency domain PPG signals and spectral templates. In some examples, the HTC unit can be configured to implement an algorithm to determine a predicted heart rate and an associated confidence measure. In some examples, heuristics can be used to determine a predicted heart rate based on the correlation curve and/or the confidence measure.

### FIELD OF THE DISCLOSURE

This relates generally to processing of a photoplethysmogram (PPG) signal and, more specifically, to using harmonic templates to determine heart rate.

### BACKGROUND OF THE DISCLOSURE

A photoplethysmogram (PPG) signal can be obtained from a pulse oximeter, which employs a light emitter and a light sensor to measure the perfusion of blood within the skin of a user. The PPG signal can be compromised by noise due to motion (e.g., acceleration) artifacts. That is, movement of the body of a user can cause the skin and vasculature to expand, sheer, and contract, introducing acceleration artifacts into the PPG signal. As a result, motion artifacts can make it difficult to effectively determine a user's heart rate.

### BRIEF SUMMARY OF THE DISCLOSURE

This relates to removing motion artifacts from the PPG signal in the frequency domain and using a harmonic template classifier (HTC) to determine heart rate. Some examples of the present disclosure utilize an accelerometer to measure movements of the user and signal processing of the PPG signal in combination with the accelerometer signal to remove unwanted artifacts in the frequency domain. For example, an acceleration mask can be generated and combined with the PPG signal to filter out acceleration contributions represented in the PPG signal. Additionally or alternatively, in some examples, an HTC unit can be configured to generate a heart rate correlation curve based on the correlation between frequency domain PPG signals and spectral templates. In some examples, the HTC unit can be configured to implement an algorithm to determine a predicted heart rate and a confidence measure associated with the predicted heart rate. In some examples, heuristics can be used to determine a predicted heart rate based on the correlation curve and/or the confidence measure.

### BRIEF DESCRIPTION OF THE DRAWINGS

FIG. 1 illustrates an exemplary block diagram of a system for determining heart rate using acceleration removal and/or harmonic templates according to examples of the disclosure.

FIG. 2 illustrates an exemplary block diagram of functional units that can be contained within or controlled by the processor of FIG. 1.

FIGS. 3A and 3B illustrate exemplary time domain and frequency domain representations of a PPG signal according to examples of the disclosure.

FIG. 4 illustrates an exemplary block diagram of the acceleration removal unit according to examples of the disclosure.

FIG. 5A illustrates an exemplary plot of frequency domain acceleration signals and a mask generated therefrom according to examples of the disclosure.

FIG. 5B illustrates an exemplary frequency domain representation of the PPG signals including motion artifacts according to examples of the disclosure.

FIG. 5C illustrates an exemplary acceleration-filtered PPG signal according to examples of the disclosure.

FIG. 6 illustrates an exemplary block diagram of the harmonic template classifier unit according to examples of the disclosure.

FIG. 7A illustrates a discrete time domain template signal for a specified heart rate according to examples of the disclosure.

FIG. 7B illustrates a spectral template generated from a discrete time domain template signal according to examples of the disclosure.

FIGS. 8A and 8B illustrate plots of correlation measures between spectral templates and a PPG signal according to examples of the disclosure.

FIG. 9 illustrates an exemplary heart rate correlation curve according to examples of the disclosure.

FIG. 10 illustrates an exemplary multi-modal heart rate correlation curve according to examples of the disclosure.

FIG. 11 illustrates an exemplary heart rate correlation curve and correlation metric according to examples of the disclosure.

FIG. 12 illustrates an exemplary plot of possible heart rate limitations based on last known or historical predicted heart rates according to examples of the disclosure.

FIG. 13 illustrates an exemplary windowing of a heart rate correlation curve according to examples of the disclosure.

FIG. 14 illustrates an algorithm for removing acceleration artifacts from a PPG signal according to examples of the disclosure.

FIG. 15 illustrates an algorithm for generating and storing spectral templates according to examples of the disclosure.

FIG. 16 illustrates an algorithm for generating a heart rate correlation curve and confidence measure according to examples of the disclosure.

FIG. 17 illustrates heuristics for determining a predicted heart rate according to examples of the disclosure.

FIG. 18 illustrates a block diagram of an exemplary system architecture that can implement the acceleration removal and/or harmonic template classifier algorithms according to examples of the disclosure.

### DETAILED DESCRIPTION OF THE DISCLOSURE

In the following description of examples, reference is made to the accompanying drawings which form a part hereof, and in which it is shown by way of illustration specific examples that can be practiced. It is to be understood that other examples can be used and structural changes can be made without departing from the scope of the disclosed examples.

A photoplethysmogram (PPG) signal can be obtained from a pulse oximeter, which employs a light emitter and a light sensor to measure the perfusion of blood within the skin of a user. However, the signal can be compromised by noise due to motion artifacts, especially those caused by acceleration. That is, movement of the body of a user can cause the skin and vasculature to expand, sheer, and contract, introducing noise into the signal. To address the presence of motion artifacts, some examples of the present disclosure utilize an accelerometer to measure movements of the user and signal processing of the PPG signal in combination with the accelerometer signal to remove unwanted artifacts in the frequency domain. For example, an acceleration mask can be generated and applied to the PPG signal to filter out acceleration contributions represented in the PPG signal. Additionally or alternatively, in some examples, a harmonic template classifier (HTC) unit can be configured to generate a heart rate correlation curve based on the correlation between frequency domain PPG signals and spectral templates. In some examples, the HTC unit can be configured to implement an algorithm to determine a predicted heart rate and a confidence measure associated with the predicted heart rate. In some examples, heuristics can be used to determine a predicted heart rate based on the correlation curve and/or the confidence measure.

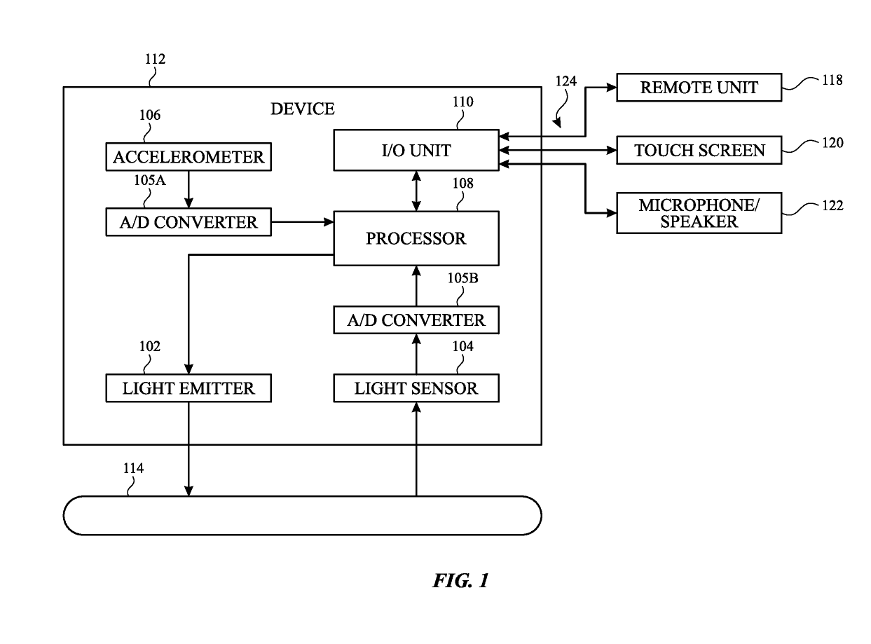

FIG. 1 illustrates an exemplary block diagram of a system for determining heart rate using acceleration removal and/or harmonic templates according to examples of the disclosure. As illustrated in FIG. 1, the block diagram can include a light emitter 102, light sensor 104, analog-to-digital converters (ADCs) 105a and 105b, accelerometer 106, processor 108 and input/output (I/O) unit 110. These components can be incorporated within a physical device 112 that can be worn or held by a user so as to secure the device to a user's skin (a limb, for example) or otherwise attached to an article of clothing worn by the user, with the light emitter 102 and light sensor 104 positioned proximate to a user's skin. Alternately, the device 112 can be entirely or partially incorporated within a smartphone or other portable device such that a user can hold the smartphone in a manner to cause the below described light beam to be reflected from the user's skin back into a light sensor positioned within the smartphone itself. A portion of the light from light emitter 102 can be absorbed by the skin, vasculature, and/or blood, among other possibilities, and a portion can be reflected back to a light sensor 104 co-located with the light emitter. The signals from the light sensor 104 can include heart rate signals due to the blood pulse wave.

Although illustrated in FIG. 1 as having only a single light emitter 102 and light sensor 104, in other examples multiple channels can be used in the system. The multiple channels can be created by increasing the number of emitter/sensor pairs, where each emitter/sensor pair can create a new channel, for example. In other examples, multiple channels can be created using different light paths from one emitter to multiple sensors (e.g., one emitter and five sensors can produce five light paths). In yet other examples, multiple channels can be created using different light paths from multiple emitters to multiple sensors (e.g., two emitters and two sensors can produce four light paths including two paths from a first emitter to each of the two sensors and two paths from a second emitter to each of the two sensors). The one or more light emitters can produce light in ranges corresponding to infrared (IR), green, amber, blue and/or red light, among other possibilities. In some examples, a light emitter can be a light emitting diode (LED) and a light sensor can be a photodiode.

The accelerometer 106 can provide time domain acceleration output signals indicative of acceleration due to movements of the user. For example, the device 112 can be worn on a user's wrist, and the accelerometer output signals can be indicative of the arm movements (i.e., arm swings) made by the user. In other examples, the accelerometer output signals can be indicative of the gait (i.e., foot strike rate) of the user. In some examples, the accelerometer can be a three-axis accelerometer providing three-dimensional acceleration outputs (e.g., three channels of acceleration outputs).

In operation, the light emitter 102 can transmit a light beam to the user's skin 114, and the light beam can be reflected by the user's skin 114 and received by the light sensor 104. The light sensor 104 can convert this light into an electrical signal indicative of the intensity thereof. This electrical signal can be in analog form and can be converted into digital form by ADC 105b. The digital signal from the ADC 105b can be a time domain PPG heart rate signal which can be fed to the processor 108. The outputs of the accelerometer 106 can also be converted to digital form using ADC 105a. The processor 108 can receive the digitized PPG signal from the light sensor 104 and the digitized accelerometer output signals from the accelerometer 106, and can process these signals to provide a heart rate (HR) output signal to the I/O unit 110. The I/O unit 110 can take the form of one or more of a storage device, a visual display, an audible annunciator, a touch screen integrated with device 112, or other output indicator. The I/O unit 110 can, under program control from the processor 108, provide historical information in visual (numeric, tabular, graphic) or audible (synthesized voice or tone) form of the detected heart rate over a period of time. As one non-limiting example, a visual graph can be displayed showing the heart rate calculated for each five minutes during a prior fixed time interval (e.g., one hour) or after an exercise period has been completed as determined by an indication thereof from the user. The I/O unit 110 can also provide, under control of the processor 108, average heart rate information or statistical information of the heart rate over a prior time period or periods. As a further example, the I/O unit 110 can provide current heart rate values as “real time” or instantaneous heart rate values displayed to the user periodically (e.g., every second) during the course of an ongoing exercise program.

The I/O unit 110 can be coupled to one or more of remote unit 118, touch screen 120 and microphone/speaker 122 or other device via wired or wireless communication links 124. The remote unit 118 can be a smart phone or other I/O device conveniently carried or worn by the user, or can be a distant computer or data server such as the user's home computer or the user's cloud storage service. The I/O unit 110 can receive input from the remote unit 118 or can receive input from the user by means of the touch screen 120 and/or the microphone/speaker 122.

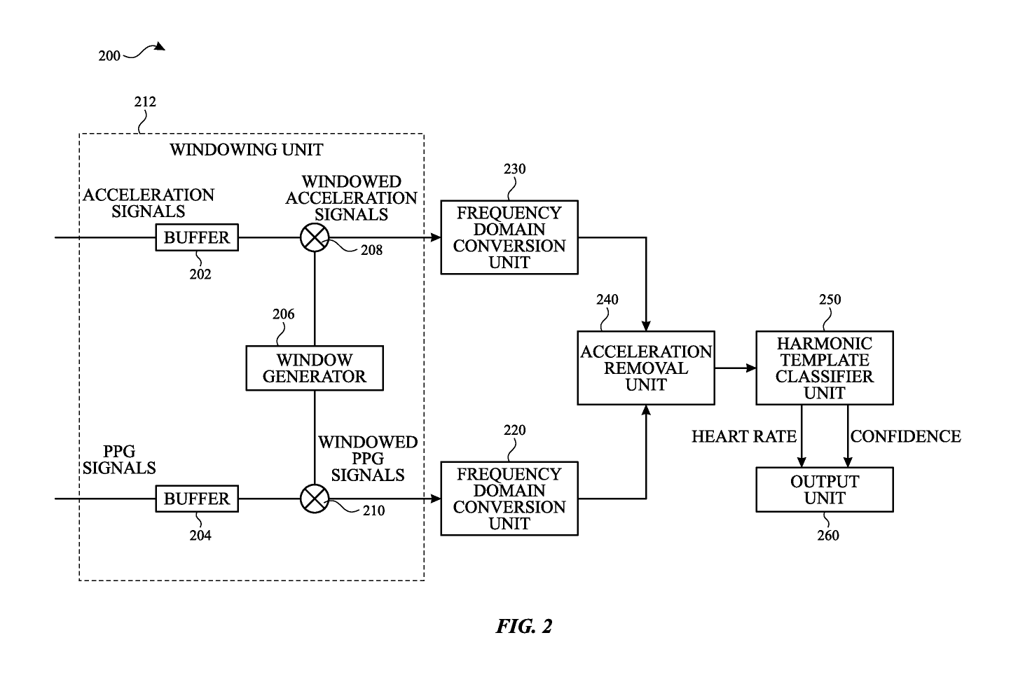

FIG. 2 illustrates an exemplary block diagram of functional units that can be contained within or controlled by the processor 108 of FIG. 1. The functional units can be implemented as discrete hardware units such as, for example, digital signal processors (DSP), application specific integrated circuits (ASIC), field programmable logic arrays (FPGA), or the like. The functional units can be combined into one or more interconnected devices. Alternatively, the functional units can be implemented in the form of software or firmware configured to operate a programmable processor. Further, the functional units can be a combination of discrete hardware, software and firmware.

Block diagram 200 includes windowing unit 212 that can generate windowed time domain PPG and acceleration signals for processing, frequency domain conversion units 220 and 230 that can convert windowed time domain PPG and acceleration signals into the frequency domain, acceleration removal unit 240 that can remove motion artifacts from the frequency domain PPG signal, harmonic template classifier (HTC) unit 250 that can determine heart rate and output unit 260 that can output the determined heart rate. The functionality and operation of the functional units in block diagram 200 are described in more detail below.

It should be understood that additional functional units can be included, such as time-domain and/or frequency domain filter units, other signal-processing units, or timing and control units. For example, a filter unit can receive time domain PPG signals including components indicative of a user's heart rate and motion artifact components. The filter unit can, for example, be a third order band-pass filter having a band-pass of 0.5-8 Hz corresponding to 30-480 bpm (beats per minute), or any other range of expected heart rate frequencies. The filter unit can remove signals that do not have frequency components that fall within or near the range expected for heart rate monitoring under various expected user conditions or activity levels. The filtering can be performed, for example, after generating windowed PPG signals, but before converting PPG signals into the frequency domain in frequency domain conversion unit 220.

Returning to the block diagram illustrated in FIG. 2, windowing unit 212 can generate windowed time domain PPG and acceleration signals. A sampling window can be established to assemble a fixed number of time aligned PPG and acceleration signal samples that can be processed by the harmonic template classifier (HTC) algorithm. In some examples, the HTC algorithm can process an eight second window of PPG and acceleration signals (e.g., from time t=0 to t=8). Although described here as an eight second window, the window can be selected to be 6-8 seconds in duration in some examples. In other examples, the range of the window can be larger, such as from 2 to 16 seconds. Several factors can contribute to determining the window size. For example, a shorter window can result in a heart rate output (e.g., average heart rate over the course of the window) that more strongly correlates to the actual heart rate conditions at the time (i.e., output can be less impacted by changes in heart rate that occurred earlier or later in time). In contrast, a larger window can result in additional samples of the PPG signal that improve the ability to detect the heart rate signal (e.g., at 60 bpm an 8 second window can contain 8 preiods of the heart rate signal).

Additionally, the system can use a sliding window to apply the HTC algorithm to samples from overlapping windows. The spacing between the overlapping windows can be 1-3 seconds in some examples (in other examples the spacing can be smaller or larger), but the example that follows will assume one second spacing. For example, a first window can look at data from PPG and accelerometer samples from time t=0 to t=8 and a second window can look at data from time t=1 to t=9. In other words, after applying the HTC algorithm to the first window, the system can discard or exclude the first second's worth of data (e.g., samples from time t=0 to t=1) and can apply the HTC algorithm to the second window including 7 seconds of data from the first window in addition to data from then next second in time (e.g., 1024 samples from time t=1 to t=9 when the sampling rate is 128 Hz). The process can be repeated such that the HTC algorithm can be applied with respect to each window (hence the sliding window). A sliding window can provide the benefit of having sufficient data to predict heart rate, but also providing an output every second, for example. Additionally, sequential outputs can be based on overlapping input samples (i.e., not independent of one another) such that the outputs are likely to be unaffected by short instantaneous changes in heart rate or noise.

Windowing unit 212 can utilize buffers 202 and 204. Buffers 202 and 204 can be first-in first-out (FIFO) buffers configured to receive time domain signal samples from the PPG and accelerometer sensors sampled by ADCs 105a and 105b. For example, ADCs 105a and 105b can have a sampling frequency of 128 Hz, although other sampling rates are possible. A 128 Hz sampling rate can produce 1024 samples for an 8 second window for each of the PPG and accelerometer signals. Buffers 202 and 204 respectively can supply the appropriate quantity of PPG and accelerometer signal samples for additional processing. In some examples, the buffers 202 and 204 can be controlled by a timing and control unit (not shown).

Window generator 206 can also include window generator 206 and multipliers 208 and 210. Window generator 206 can generate a windowing function so that the harmonic template classifier algorithm can operate on a fixed number of samples. The function generated by the window generator 206 can be mixed with the acceleration signals from buffer 202 at multiplier 208 to generate windowed acceleration signals. Similarly, the function generated by the window generator 206 can be mixed with the PPG signals from buffer 204 at multiplier 210 to generate windowed PPG signals.

In some examples, the windowing function can be a unit step function such that all portions of the signal outside the window of interest can be multiplied by zero and the portions of the signal inside the window of interest can be multiplied by 1. Using a unit step function, however, can cause significant distortion due to the abrupt change at the edges when converting to the frequency domain. Alternatively, rather than using the unit step function applied by the window generator 206 and multipliers 208 and 210, the buffers 202 and 204 can be controlled to output data corresponding to the window of interest (i.e. exactly 1024 data samples for each of the PPG and acceleration signals assuming an 8 second window and a 128 Hz sampling rate).

In other examples, window generator 206 can generate a Hanning-window function. A Hanning-window function can be zero at the edges to remove the portions of the signal outside the window of interest completely and then gradually increase to 1 in the center of the window of interest. Using a Hanning-window or similar windowing function can reduce undesirable frequency domain artifacts resulting from abrupt endpoint transitions of the time domain PPG and acceleration signals.

The windowed PPG and acceleration signals can be fed into frequency domain conversion units 220 and 230. The frequency domain conversion units 220 and 230 can implement an algorithm to convert time domain signals into frequency domain signals (e.g., using a discrete Fourier transform (DFT)). In some examples, the conversion algorithm can be the efficient fast Fourier transform (FFT), although other algorithms can be used. The FFT for both frequency domain conversion units 220 and 230 can, for example, be carried out by the same hardware, firmware and/or software and can be computed in parallel or can be time multiplexed to receive the windowed time domain PPG and acceleration signals. In some cases, the FFT can be performed by existing dedicated hardware or firmware for performing an FFT. The output of the frequency domain conversion units 220 and 230 can be real value outputs indicative of the magnitudes (and phases) of the frequency coefficients for the PPG and acceleration signals during the window of interest. Thus, the output of frequency domain conversion unit 220 can be a frequency domain representation of the PPG signals and the output of frequency domain conversion unit 230 can be a frequency domain representation of the acceleration signals.

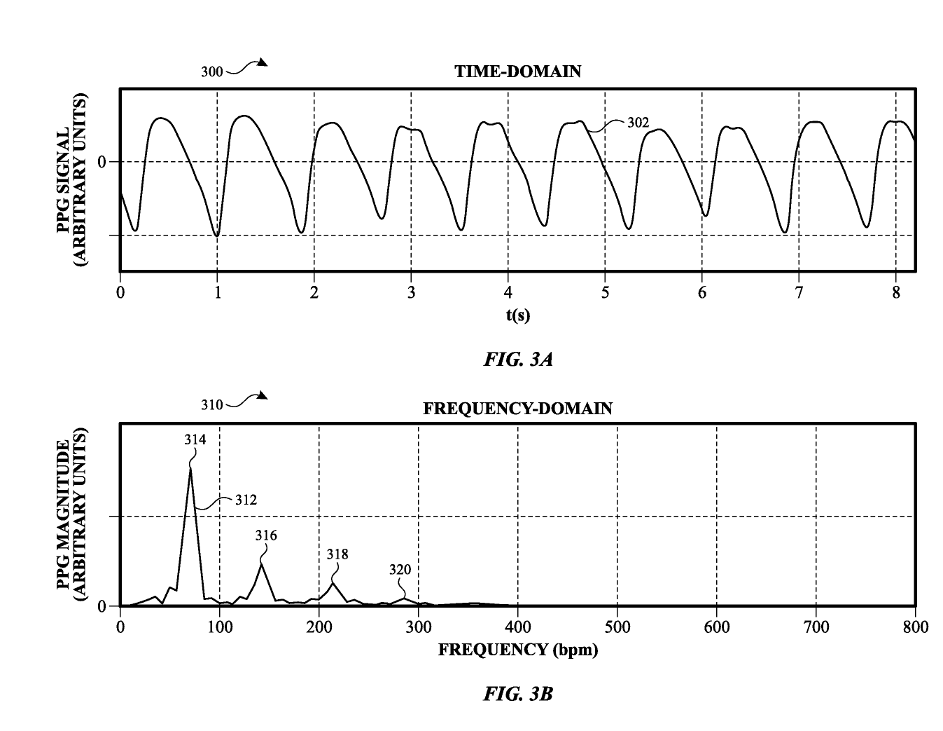

FIGS. 3A and 3B illustrate exemplary time domain and frequency domain representations of a PPG signal according to examples of the disclosure. As illustrated in plot 300 in FIG. 3A, a time domain PPG signal 302 can have a periodic structure, similar to a sinusoid. The PPG signal can correspond to a stationary user such that no motion artifact can be introduced into the PPG signal. The time domain PPG signal 302 can be sampled during a window of interest to generate a discrete time domain PPG signal as described above (e.g., 1024 samples for an 8 second window with a 128 Hz sampling rate). The frequency domain conversion algorithm can be applied to the discrete time domain PPG signals to generate magnitudes (and phases) of the frequency coefficients. FIG. 3B illustrates a plot 310 of the frequency domain PPG signal coefficients 312 corresponding the time domain PPG signal 302 of FIG. 3A. The frequency domain plot 310 can have frequency represented on the x-axis and magnitude represented on the y-axis. For a periodic (or near periodic) signal, like time domain PPG signal 302, the frequency domain PPG signal coefficients 312 can have a harmonic structure. As illustrated in FIG. 3B, the fundamental (i.e., first harmonic) 314 appears at about 70 bpm (beats per minute) and has the largest amplitude. Second harmonic 316 can appear at about 140 bpm with a smaller amplitude than the fundamental 314. Third harmonic 318 can appear at about 210 bpm with a smaller amplitude than the second harmonic 316. Fourth harmonic 320 can appear at about 280 bpm with a smaller amplitude than the third harmonic 318. The harmonic structure can be reflected in the spacing between the harmonics of 70 bpm, the fundamental frequency. The amplitude corresponding to the magnitude of the coefficient can decline rapidly (e.g., exponentially) and thus, although additional harmonics can occur, they can be negligible as most of the energy (e.g., more than 90%) of the PPG signal can be captured in the first four harmonics.

Although FIGS. 3A and 3B illustrate a frequency domain conversion of the PPG signals, the frequency domain conversion unit 230 can perform a similar operation on the acceleration signals. In some examples, acceleration signals can be the output of a 3-axis accelerometer. Frequency domain conversion unit 230 can perform the frequency domain conversion on each of the 3 separate input channels and then can sum the magnitudes to produce one frequency representation of the acceleration signals. In other examples, the accelerometer can output a single signal stream (e.g., representative of the acceleration contribution from three dimensions) or the 3 accelerometer output channels can be summed in the time domain before the frequency domain conversion.

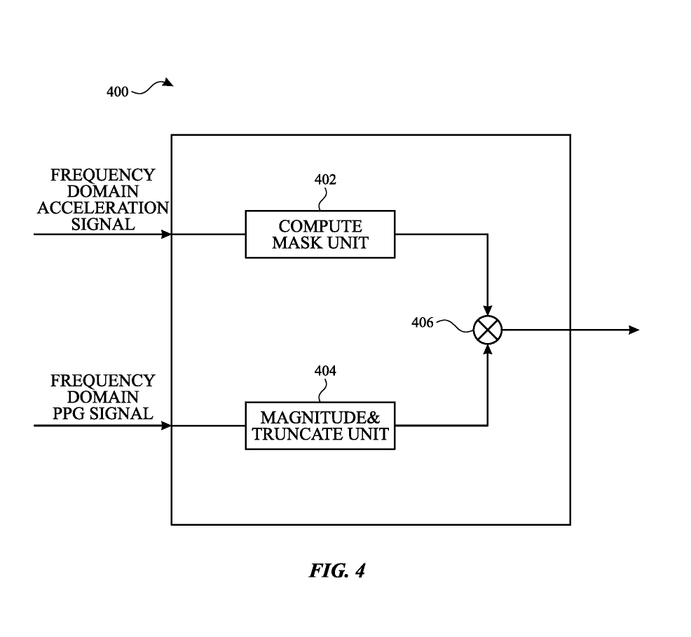

FIG. 4 illustrates an exemplary block diagram of the acceleration removal unit according to examples of the disclosure. Acceleration removal unit 400 can include a compute mask unit 402, a magnitude and truncate unit 404, and a spectral domain multiplier unit 406.

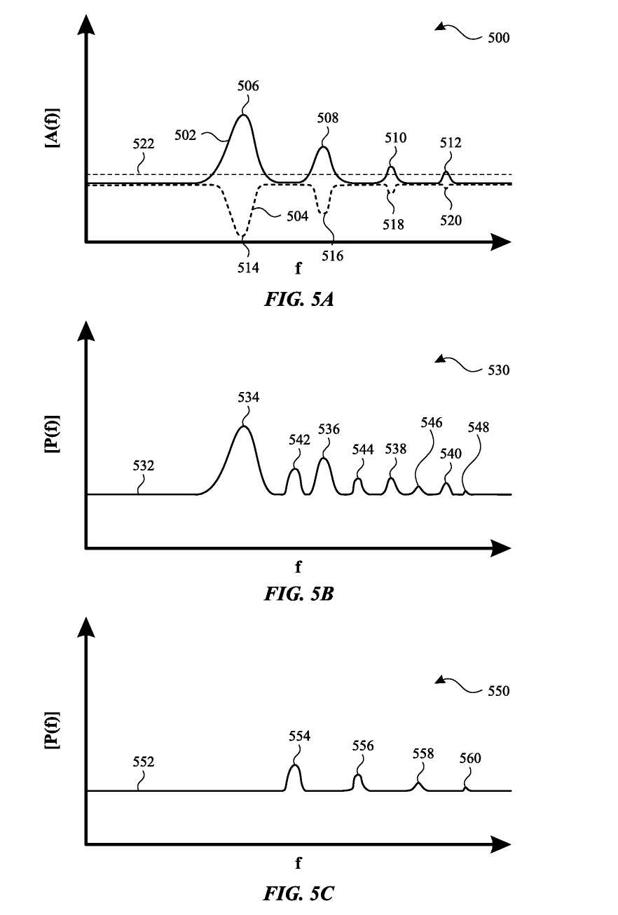

Compute mask unit 402 can accept a frequency domain representation of the acceleration signals as an input to generate a mask used to filter out the acceleration components from the PPG signal. FIG. 5A illustrates an exemplary plot of frequency domain acceleration signals and a mask generated therefrom according to examples of the disclosure. Plot 500 of FIG. 5A illustrates magnitude of the frequency domain acceleration signal coefficients on the y-axis and frequency on the x-axis. The frequency domain acceleration signal coefficients 502 can be unity at frequencies with no acceleration (i.e., 1 gravitational unit of acceleration) and can have a harmonic structure caused by periodic motion represented by harmonics 506, 508, 510 and 512 separated from one another by the fundamental frequency. A mean of the magnitudes of the acceleration signal coefficients can be calculated as illustrated by curve 522. A mask 504 can be generated using the mean of the magnitudes. For example, the mask can be computed to be the maximum of the inverse of the magnitude of the acceleration coefficient or unity for all acceleration coefficients greater than the mean. Mathematically, the mask computation can be represented as in equation (1):

$$M ( f ) = max \{ \frac{1}{\vert A ( f ) \vert} , 1 \} , \\ for \vert A ( f ) \vert > \vert \overline{A ( f )} \vert \qquad ( 1 )$$

where M(f) can represent the mask as a function of frequency, |A(f)| can represent the magnitude of the acceleration signal coefficients, and |̅A̅(̅f̅)̅|̅ can represent the mean of the magnitude of the acceleration coefficients. Thus, in the illustration of FIG. 5A, the mask can be unity at all locations where the magnitude of the coefficients is below the mean and can be equal to the inverse of the magnitude at locations where the magnitude of the coefficients is above the mean. The inverted peaks 514, 516, 518 and 520 in mask 504 can correspond to peaks of harmonics 506, 508, 510 and 512 in the frequency domain acceleration signal coefficients. Mask 504 can be used to filter out the motion artifacts represented in the PPG signal as discussed in more detail below.

The magnitude and truncate unit 404 can be used to prepare the frequency domain PPG signals for filtering. The frequency domain conversion discussed above can generate complex coefficients. Magnitude and truncate unit 404 can generate a magnitude representation of the coefficients. In other examples, the frequency domain conversion unit 220 can output a magnitude representation of the coefficient rather than generating a magnitude representation in the magnitude and truncate unit 404. Additionally, magnitude and truncate unit 404 can truncate the frequency domain representation to exclude coefficients outside the range of interest. For example, an upper bound of possible heart rates can be 220 bpm (3.67 Hz) and a fourth harmonic can occur at 880 bpm (14.67 Hz). Thus, for the frequencies of interest (e.g., 35 bpm to 220 bpm), the frequency information above a threshold frequency (e.g., 20 Hz including a generous margin) can be ignored. Magnitude and truncate unit 404 can truncate the frequency domain PPG signal to ignore the coefficients above the threshold frequency. Similarly, compute mask unit 402 can also generate a magnitude representation of the acceleration signals (or receive a magnitude representation from frequency domain conversion unit 230) and truncate the acceleration signals to the frequency of interest before (or after) computing the acceleration mask.

FIG. 5B illustrates an exemplary frequency domain representation of the PPG signals including motion artifacts according to examples of the disclosure. Plot 530 of FIG. 5B illustrates magnitude of the frequency domain PPG signal coefficients on the y-axis and frequency on the x-axis corresponding to the same window as plot 500 of FIG. 5A. The frequency domain PPG signal coefficients 532 can include heart rate contributions and acceleration contributions. The heart rate contributions and the acceleration contributions can have harmonic structure. For example, harmonics 542, 544, 546 and 548 can correspond to periodic heart rate contributions. Harmonics 534, 536, 538 and 540 can correspond to the acceleration contributions caused by periodic motion and corresponding to harmonics 506, 508, 510 and 512 in FIG. 5A. As discussed above, magnitude and truncate unit 404 can remove high frequency coefficients of the PPG signal leaving only harmonics in the frequency range of interest.

Referring back to FIG. 4, the outputs of compute mask unit 402 (i.e., the acceleration mask) and magnitude and truncate unit 404 (i.e., truncated PPG signals) can be input into spectral domain multiplier unit 406. The spectral domain multiplier unit 406 can multiply the acceleration mask and truncated PPG signals to filter out the acceleration contribution from the PPG signals. In effect, the acceleration mask can operate as a matched filter matched to the frequencies corresponding to the acceleration contributions. Thus, the portions of the acceleration mask with a value unity can leave the truncated PPG signals unchanged, but the portions of the acceleration mask with inverted peaks can reduce or completely remove the corresponding peaks in the PPG signal corresponding to the acceleration.

The output of the spectral domain multiplier unit 406 can be the acceleration-filtered PPG signal. FIG. 5C illustrates an exemplary acceleration-filtered PPG signal according to examples of the disclosure. Plot 550 of FIG. 5C illustrates magnitude of the acceleration-filtered PPG signal coefficients on the y-axis and frequency on the x-axis corresponding to the same window as plots 500 and 530 of FIGS. 5A and 5B. The acceleration-filtered PPG signal coefficients 552 can include heart rate contributions with a harmonic structure. For example, harmonics 554, 556, 558 and 560 can correspond to periodic heart rate contributions and correspond to harmonics 542, 544, 546 and 548 of the frequency domain PPG signal coefficients 532 in FIG. 5B. The acceleration contribution harmonic structure of the frequency domain PPG signal coefficients can be removed. The acceleration-filtered PPG signal can be output from the acceleration removal unit 240 and input into HTC unit 250.

Although the acceleration removal unit 240 can improve performance by removing motion artifacts, the unit can be optional and not necessary for the HTC algorithm described below in more detail. In cases with little or no motion, for example, the acceleration removal unit 240 can remove few or no motion artifacts. In such a case, the acceleration removal operation can be bypassed (if implemented at all), though performing acceleration removal in the absence of motion can effectively leave the PPG signal unchanged. Additionally, although the example of FIG. 5B illustrates that heart rate and acceleration harmonics can occur at different frequencies, in some examples, known as cardio-locomotor synchronization (CLS), the acceleration harmonics and heart rate harmonics in the PPG signal can overlap partially or completely. In CLS examples, alternative acceleration removal algorithms can be used to remove the motion artifacts. For example, the acceleration removal unit can implement other acceleration removal techniques such as those described in co-pending U.S. patent application (Maani et al., ATTORNEY DOCKET NUMBER 10684-21202.00) filed on the same day as the instant application and assigned to the common assignee of the instant application and/or U.S. patent application (Waydo, ATTORNEY DOCKET NUMBER 10684-21201.00) filed on the same day as the instant application and assigned to the common assignee of the instant application. These co-pending applications are also incorporated by reference herein in their entirety for all purposes.

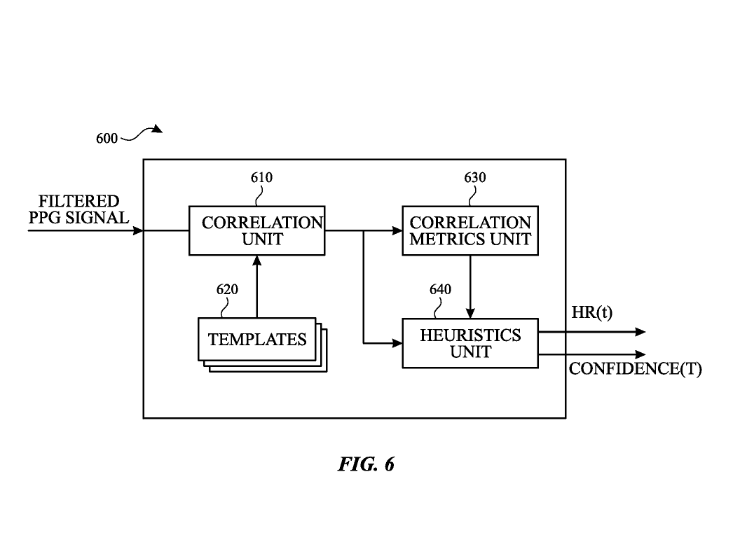

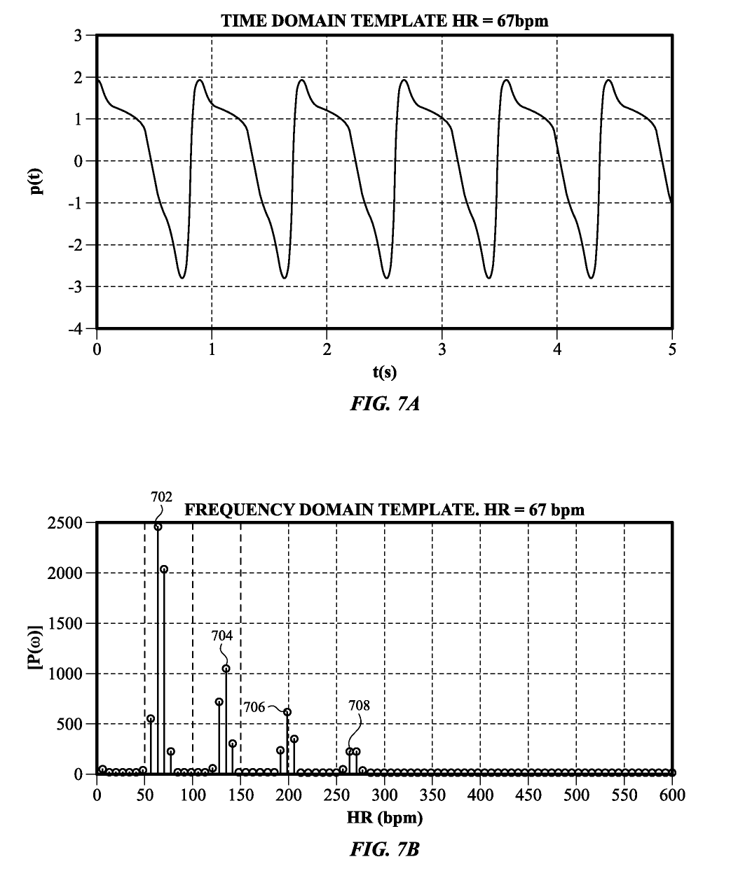

FIG. 6 illustrates an exemplary block diagram of the HTC unit according to examples of the disclosure. HTC unit 600 can include a correlation unit 610, template library 620, correlation metrics unit 630 and heuristics unit 640. A set of spectral templates can be generated for multiple candidate heart rates and stored in template library 620. For each candidate heart rate, a discrete time domain template signal can be generated by a synthetic waveform generator according to equation (2):

$$p ( n , {θ}_{r} ) = {\sum }_{m = 1}^{4} {a}_{m} sin [ m {ω}_{0} ( r ) {nT}_{s} ] + {\sum }_{m = 1}^{4} {b}_{m} cos [ m {ω}_{0} ( r ) {nT}_{s} ] \\ n ∈ \{ 0 , 1 , … , N - 1 \} \qquad ( 2 )$$

where p(n, θᵣ) can represent a periodic heart rate signal value for sample n and heart rate frequency θᵣ, aₘ and bₘ can represent scaling coefficients, m can represent a frequency multiplier, ω₀(r) can be the angular frequency for a given heart rate (e.g., equal to 2ηr/60 where r can be in bpm), and Tₛ can be the chosen sampling period (e.g., measured in seconds). The discrete time domain template signal generated by equation (2) can have a composite sinusoidal structure made up of four separate sinusoidal components, although a different number of sinusoidal components can be possible. Four components can be desirable because a four harmonic spectral signal can effectively represent the pulse shape of an undistorted PPG signal. FIG. 7A illustrates a discrete time domain template signal for a specified heart rate according to examples of the disclosure. As illustrated in FIG. 7A, the discrete time domain template signal for a heart rate of 67 bpm can have a periodic shape and spacing between the peaks of 0.9 seconds.

The function generated using equation (2) can be represented more compactly in the frequency domain (e.g., using 8 real numbers along with a frequency parameter) and can have unique spectral line signatures. A spectral template can be generated by converting the discrete time domain template signal into the frequency domain, e.g., by performing a DFT for a suitable window. Mathematically, this can be represented as in equation (3):

$$P ( k , {θ}_{r} ) = {\sum }_{n = 0}^{N - 1} {ω}_{n} p ( n ) {W}_{N}^{kn} \\ k ∈ \{ 0 , 1 , … , N - 1 \} \qquad ( 3 )$$

where P (k, θᵣ) can represent the frequency domain heart rate coefficient for sample k and heart rate frequency θᵣ, ωₙ can represent the chosen window, p(n) can represent the amplitude of the time domain heart rate signal sample and W_{N} can represent the phase component (i.e., e^{-2πi/N}). FIG. 7B illustrates a spectral template generated from a discrete time domain template signal according to examples of the disclosure. As illustrated in FIG. 7B, the spectral template signal for a heart rate of 67 bpm can have four harmonics (corresponding to the four components in the discrete time domain template signal) including fundamental 702 at approximately the fundamental frequency of 67 bpm, second harmonic 704 at approximately 134 bpm, third harmonic 706 at approximately 201 bpm and fourth harmonic 708 at approximately 268 bpm. It should be understood that although waveforms can be generated using equations (2) and (3), in other examples, custom waveforms can also be generated that can be fitted to a measured heart rate signal.

It should be understood that the spectral templates can be generated by the system in some examples, or alternatively the spectral templates can generated by a different system and the template data stored in a library (e.g., a memory) in the system. Spectral templates can be generated for possible candidate heart rates. The range of candidate heart rates can be 35 bpm to 220 bpm to capture the reasonable range of expected heart rates (with some considerable margin), although other ranges are possible. In order to reduce the amount of spectral data stored, some spectral data can be ignored (e.g., truncating the spectral template). For example, at an expected heart rate of 220 bpm, the fourth harmonic occurs at 880 bpm, the higher frequency coefficients can be outside the frequency range of interest as discussed above. In some examples, the spectrum of stored values can be limited to reduce the storage requirements. For example, useful coefficients of P(k) can be defined by equation (4):

$$k : 0 \leq k \leq N ( \frac{{f}_{l}}{{F}_{s}} ) \qquad ( 4 )$$

where k can represent a spectral sample, N can represent the number of time-domain samples contained by a transform window, fₗ can represent the spectrum of interest, and Fₛ can represent the sampling rate. Thus, in an example with N=4096 samples, fₗ=20 Hz (i.e., 1200 bpm), and Fₛ=500 samples per second, the number of useful coefficients can be limited to k<164.

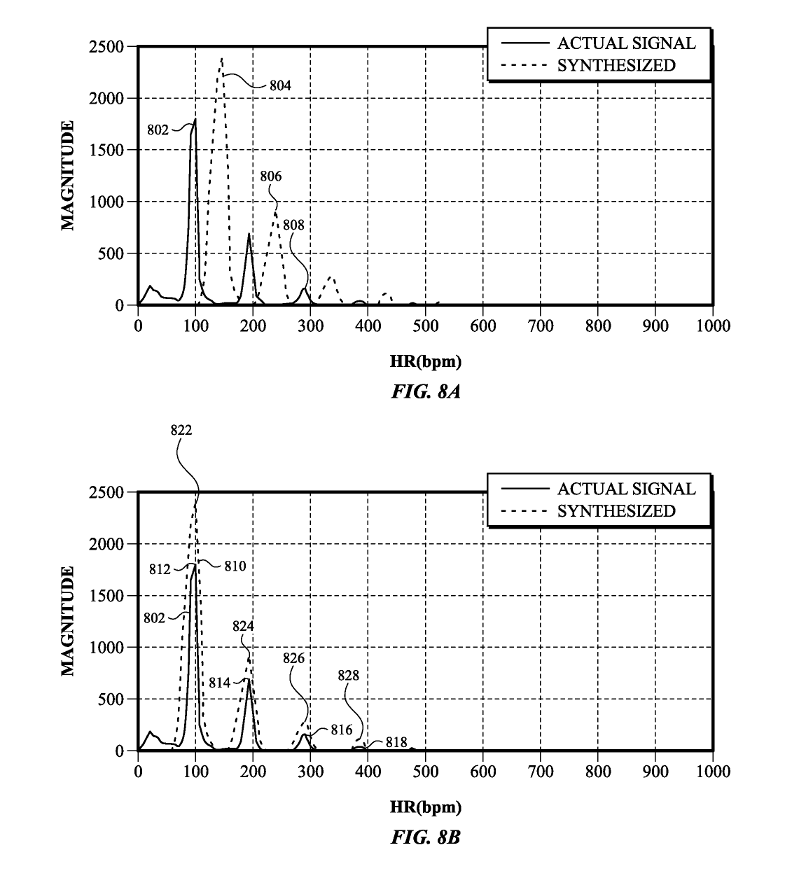

Correlation unit 610 can determine the correlation between the acceleration-filtered PPG signal with spectral templates for given heart rates. FIGS. 8A and 8B illustrate plots of correlation measures between spectral templates and a PPG signal according to examples of the disclosure. As illustrated in FIG. 8A, PPG signal 802 can have a harmonic structure with four identifiable harmonics and a fundamental frequency of approximate 98 bpm Likewise the spectral template signal 804 can have a four harmonic structure and a fundamental frequency of approximately 140 bpm. PPG signal 802 and spectral template signal 804 can be mostly uncorrelated (i.e. have a low correlation value) as indicated by the mostly separate peaks. Only peak 806 corresponding to the second harmonic of the spectral template signal 804 overlaps with the third harmonic 808 of PPG signal 802, and even though there can be overlap, the magnitudes can be considerably different because the peaks correspond to different harmonics of their respective signals. FIG. 8B, in contrast to FIG. 8A, illustrates a high correlation between the PPG signal and a different frequency spectral template. As illustrated in FIG. 8A, PPG signal 802 can still have harmonic structure with a fundamental frequency of approximately 98 bpm. Spectral template 810 can likewise have a four harmonic structure and a fundamental frequency of approximately 98 bpm. Thus, fundamentals 812 and 822 of the PPG signal 802 and spectral template 810 can occur at the same frequency. Similarly, harmonics 814, 816 and 818 of PPG signal 802 can occur at the same frequency as harmonics 824, 826 and 828 of the spectral template 810. The overlapping harmonics of FIG. 8B can be representative of high correlation between the PPG signal 802 and the spectral template 810, and therefore the heart rate of spectral template 810 can be a candidate for the actual heart rate as described in more detail below.

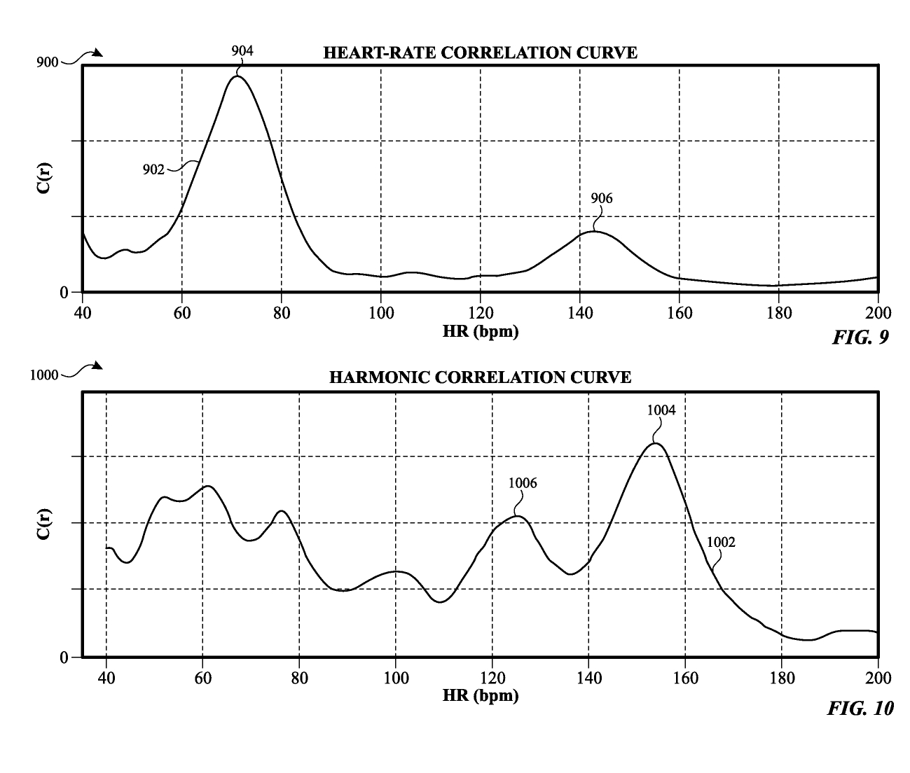

Correlation unit 610 can generate a heart rate correlation curve based on the determined correlation between the PPG signal and spectral templates for given heart rates. FIG. 9 illustrates an exemplary heart rate correlation curve according to examples of the disclosure. Plot 900 illustrated in FIG. 9 includes heart rate correlation curve 902 with a correlation value associated with the correlation between the PPG signal and the spectral template signal for each heart rate. The heart rate correlation curve 902 includes a peak 904 at approximately 72 bpm indicative of the maximum correlation (and the likely prediction of the actual heart rate). The heart rate correlation curve 902 also includes a smaller peak 906 (local maximum) at approximately 144 bpm indicative of a harmonic. Due to the frequency spacing of the harmonic structure for the PPG signal and spectral template signals, it can be common to have a local maximum harmonic of the maximum correlation fundamental.

The maximum correlation (and harmonic) peaks in heart rate correlation curve of FIG. 9 can be distinct and identify a predicted heart rate close to the actual heart rate (e.g., 72 bpm). In some examples, the heart rate correlation curve can be more ambiguous about the predicted heart rate. One reason for the ambiguity can be unfiltered motion artifacts or other noise sources, which can be correlated with spectral templates. FIG. 10 illustrates an exemplary multi-modal heart rate correlation curve according to examples of the disclosure. Plot 1000 illustrated in FIG. 10 includes heart rate correlation curve 1002 with a correlation value associated with the correlation between the PPG signal and the spectral template signal for each heart rate. The heart rate correlation curve 1002 includes a peak 1004 at approximately 154 bpm indicative of the maximum correlation, and peal 1006 (local maximum) at approximately 124 bpm with relatively high correlation. In some cases the maximum correlation can correspond to the frequency of the motion artifact or noise rather than the true heart rate. As a result, additional correlation metrics and heuristics can be necessary to determine a prediction for the actual heart rate as described below in more detail.

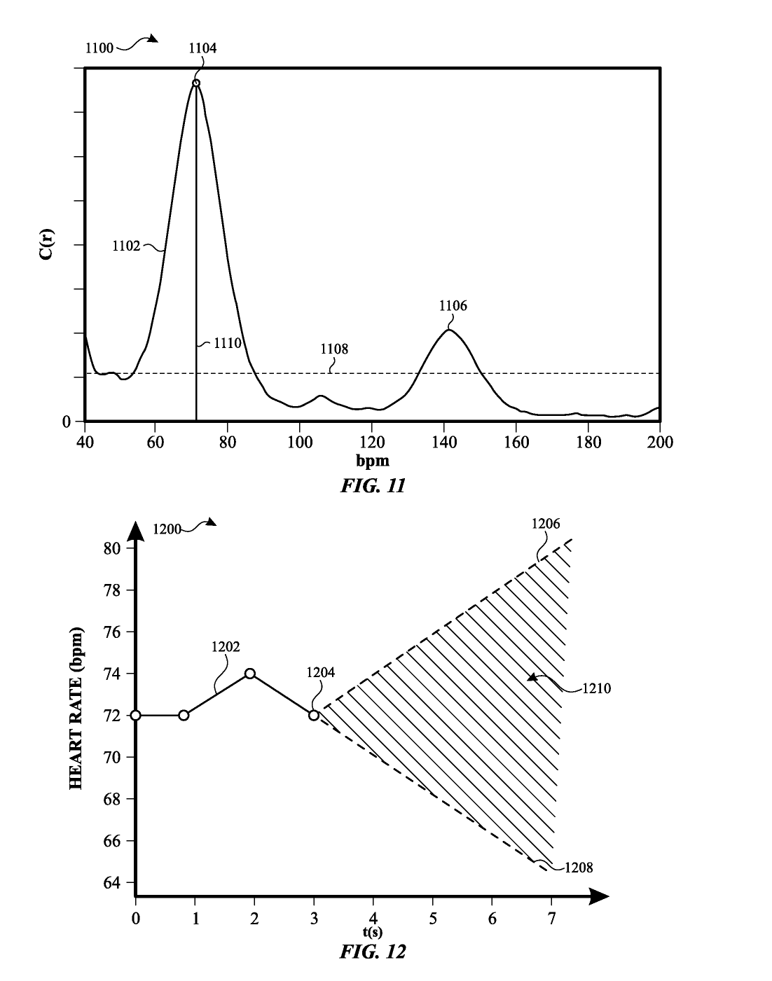

Correlation metrics unit 630 can receive the output of the correlation unit (e.g., the correlation information represented in the heart rate correlation curve) as an input to generate metrics used to determine a confidence measure for a predicted heart rate. FIG. 11 illustrates an exemplary heart rate correlation curve and correlation metric according to examples of the disclosure. Plot 1100 illustrated in FIG. 11 includes heart rate correlation curve 1102 with peak 1104 at approximately 72 bpm indicative of the maximum correlation, and peak 1106 at approximately 144 bpm indicative of a harmonic. Correlation metrics unit 630 can calculate a mean correlation value and a confidence measure that compares the maximum value to the mean value. Mathematically, the confidence measure can be expressed as in equation (5):

$$Confidence = {K}_{0} \frac{max ( C ( r ) )}{mean ( C ( r ) )} \qquad ( 5 )$$

where K₀ can represent a scaling coefficient, max(C(r)) can represent the maximum correlation value and mean(C(r)) can represent the mean of the correlation values. In other words, the larger a maximum correlation value relative to the mean correlation value can indicate a high confidence. Thus, in the example of FIG. 11, the mean correlation value 1108 can be fairly low and the correlation value 1110 at peak 1104 can be quite large relative to the mean, producing a high confidence measure that the predicted heart rate can be at approximately 72 bpm. In some examples, a confidence measure can be calculated for each peak (local maximum) in the heart rate correlation curve 1102. The correlation value at peak 1106 can be small relative to the mean (compared with the correlation value at peak 1104) and therefore have a reduced confidence measure that the predicated heart rate can be at approximately 144 bpm. Using a confidence measure and a confidence measure threshold rather than a magnitude threshold to determine a predicted heart rate can be advantageous in that it avoids complications in the case of multiple peaks that exceed the magnitude threshold. In other examples, the correlation metric can compare peaks with a median of the correlation curve rather than the mean of the correlation curve to generate a confidence measure.

In other examples, the correlation metrics can be based on PPG and/or acceleration signals. For example, one correlation metric can be based on the closeness of peaks in the unfiltered frequency domain PPG signal. Small distances between peaks can be indicative of a CLS condition and reduced effectiveness of the acceleration removal. As a result, the reduced distances between peaks can correspond to a reduced confidence measure. In contrast, a large separation between peaks can provide for effective removal of acceleration peaks and correspond to an increased confidence measure. In another example, the magnitude of the accelerometer data can be a used to generate a confidence measure. High magnitude acceleration can introduce broadband noise that can interfere with the harmonic structure. Large accelerations can upset the regularity of blood flow and can introduce dynamic changes in tissue structure, which can interfere with the PPG signal. Thus, a large magnitude acceleration can correspond to a reduced confidence measure. Additional correlation metrics can be determined in correlation metrics unit 630 as described below (though in some examples some metrics can be calculated in the heuristics unit 640).

Heuristics unit 640 can receive the output of the correlation unit (e.g., the correlation information represented in the heart rate correlation curve) as well as the output of the correlation metrics unit 630 as inputs to determine a predicted heart rate and optionally an output confidence measure. The heuristics unit 640 can implement algorithms that take advantage of physiological properties of heart rate. For example, heart rate can gravitate toward a resting heart rate, especially when physical demands such a motion decreases or stops (e.g., accelerometer magnitude declines). Likewise, heart rate can increase when motion increases after a period of little or no motion. Additionally, the heart rate process can exhibit strong Markhov properties such that the probability of the next heart rate can be strongly dependent upon the last known heart rate. Additionally, the cardiovascular control center located within the human brain can behave in a fairly predictable manner. Other considerations can also be included to provide improved heuristics for determining a predicted heart rate.

One exemplary heuristic can be to compare the confidence measure for the next predicted heart rate with a threshold confidence measure. If the next predicted heart rate cannot be determined with a confidence measure in excess of the threshold, in some examples, the heuristics unit can output the last predicted heart rate value known with sufficient confidence (i.e., meeting or exceeding the threshold). Additionally, the confidence measure associated with the output can be reduced from the confidence measure of the previous output. Reducing the confidence measure can reflect the failure of the new confidence measure to exceed the threshold. In some examples, the predicted heart rate output when the new predicted heart rate fails to reach or exceed the confidence measure threshold can be based on the last known value alone. In other examples, the new predicted output can be based on an average of a number of historical values (e.g., last three predicted values with high confidence), a median or mode of a number of historical values, or some other metric.

Another exemplary heuristic can be to use the confidence measure(s) as an indicator of modality in the heart rate correlation curve. For example, in the heart rate correlation curve of FIG. 11, the confidence measure of peak 1104 can exceed a confidence measure threshold whereas the confidence measure of peak 1106 can fail to reach or exceed the confidence measure threshold. The heuristics unit 640 (or the correlation metrics unit 630) can determine that the heart rate correlation curve in the example of FIG. 11 can have a single mode because only one confidence measure exceeds the confidence measure threshold. In the example of multi-modal heart rate correlation curve of FIG. 10, however, both of peaks 1004 and 1006 can exceed the confidence measure threshold and the modality can be determined to be bi-modal. The heuristics unit 640 can use the modality information to help predict a heart rate. In a single mode case (as illustrated in FIG. 11), the predicted heart rate can be selected based on the peak 1104 (e.g., the single mode peak exceeding the confidence measure threshold). Thus, the predicted heart rate of 72 bpm can be output and the associated confidence measure can be output as well. In contrast, in a multi-modal example (e.g., as illustrated in FIG. 10), additional heuristics can be performed before determining which of peaks 1004 and 1006 can be indicative of the predicted heart rate.

Another exemplary heuristic can be based on last known or historical heart rate values. For example, the heuristics unit 640 can store or access (e.g., from output unit 260) a history of last known heart rate values. As discussed above, the probability of the next heart rate can be strongly dependent upon the last known heart rate because physiologically, heart rate can tend to change slowly. Thus, if a last heart rate predicted can be 72 bpm, it can be unlikely that the next heart rate predicted can be 120 bpm (e.g., a change of 48 bpm per second). It can be more likely that a change of heart rate can occur within a tolerance of the last known or historical heart rate predictions. FIG. 12 illustrates an exemplary plot of possible heart rate limitations based on last known or historical predicted heart rates according to examples of the disclosure. Plot 1200 illustrated in FIG. 12 can include historical predicted heart rate values 1202 with high confidence measures. The last known predicted heart rate value 1204 at t=3 can be at approximately 72 bpm. Curves 1206 and 1208 can bound an area 1210 of possible future heart rate values, which can be referred to as the cone of reachability. In other words, the cone of reachability can set limits on future predicted heart rate values. The slope of curves 1206 and 1208 can be set based on physiological constraints, e.g., no more than ±2 bpm/second. For example, at time t=4, the predicted heart rate can be between 70 bpm and 74 bpm (±2 bpm from last known high confidence heart rate of 72 bpm). Similarly, assuming no new predicted heart rate value can be determined with sufficient confidence at t=4, the cone of reachability limits the predicted heart rate at t=5 to between 68 bpm and 76 bpm (±4 bpm from last known high confidence heart rate of 72 bpm). Note that in the latter determination, the last known value can still be at t=3, i.e., the last value predicted with a confidence measure in excess of a confidence threshold. Predicted heart rates from the correlation unit 610 and correlation metrics unit 630 outside the cone of reachability can be determined to have a reduced confidence measure or can be ignored, and instead the new predicted heart rate can be assumed to have a different heart rate (e.g., the last known value with high confidence measure).

One advantage of using the cone of reachability heuristic can be to suppress disruptors. Sudden movements can give rise to broadband energy that can effectively upset the time-local harmonic structure. The cone of reachability can be used to restrict the predicted heart rate to a small range around the last known heart rate with high confidence to avoid misdirected predictions of heart rate. In some examples, the mechanism to achieve the cone of reachability can be a time-varying filter. For example, a time-varying filter can be implemented as described mathematically in equation (6):

y(n)=a(n)x(n)+[1−a(n)]y(n−1)

a(n)=K·confidence(n) (6)

where y(n) can represent the output of the next heart rate prediction, a(n) can be a scaled measure of confidence for the current heart rate prediction, x(n) can represent the current heart rate prediction, y(n−1) can represent the last known heart rate prediction, K can represent a scaling constant, and confidence(n) can represent the confidence measure for the current heart rate prediction. In other words, the next heart rate prediction can be based on a weighted contribution of the confidence measure of the current heart prediction as well as a weighted contribution of the last known heart rate prediction. In other examples, rather than using the last known heart rate, the cone of reachability can be based on a statistical measure of historical heart rate values.

Another exemplary heuristic can be based on a centroid of the correlation curve. A frequency centroid of the correlation curve can be determined either in correlation metrics unit 630 or in heuristics unit 640 weighted based on the correlation value. In some examples, two peaks in the correlation curve can be located within the cone of reachability, and computing the centroid can give an indication of whether the next heart rate prediction can be the same heart rate as the previous prediction, an increased heart rate prediction or a decreased heart rate prediction. For example, two peaks in the correlation curve can be located at 71 bpm and 75 bpm, within the ±4 bpm of a last known heart rate of 72 bpm. If the centroid of the correlation curve can result at 74 bpm, the predicted heart rate can be 74 bpm. If the centroid of the correlation curve can result at 72 bpm, the predicted heart rate can be 72 bpm. In other examples, the centroid can be calculated in order to attempt to find a predicted heart rate within the cone or reachability when no peaks appear within the cone of reachability.

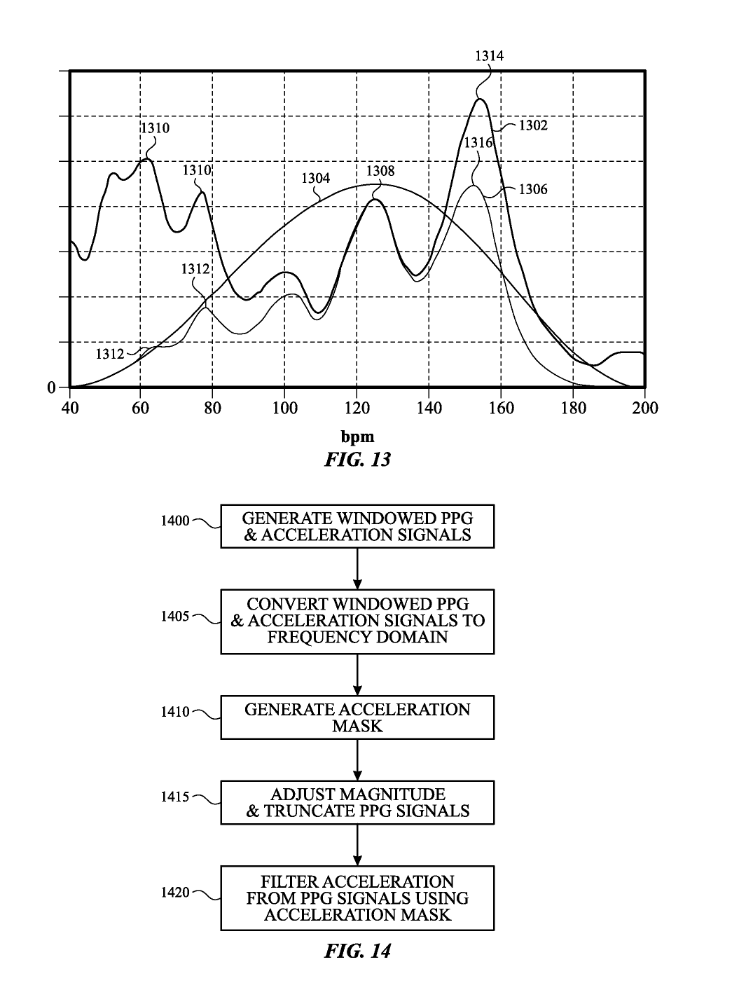

Another exemplary heuristic can be using a pass-band window to suppress some modes in the heart rate correlation curve. Periodic motion can give rise to nuisance in-band distractor modes that interfere with heart rate prediction. A pass-band window centered on the correlation curve at the frequency of the last heart rate prediction can be used to suppress distractor modes. For example, a Hanning or Hamming window can applied to the heart rate correlation curve to better reveal peaks corresponding with more likely heart rates while progressively limiting the impact of peaks far away from the last known heart rate. Such windowing can be important to suppress distractors, particularly when used along with a centroid heuristic, for example. FIG. 13 illustrates an exemplary windowing of a heart rate correlation curve according to examples of the disclosure. As illustrated in FIG. 13, a windowing function (e.g., a Hanning window) 1304 can be applied to heart rate correlation curve 1302. The windowing function 1304 can be centered around the peak 1308 in the heart rate correlation curve 1302 corresponding to the last known heart rate (e.g., 125 bpm). Windowed correlation curve 1306 can represent the output of applying windowing function 1304 to heart rate correlation curve 1302. Lower frequency peaks 1310 (perhaps sub-harmonics of peaks 1308 and 1314) distant from the center of the window can be largely removed from the windowed correlation curve 1306 as illustrated by corresponding peaks 1312. Peak 1308 at the center of the window can be largely unaffected by the windowing operation. Peak 1314, can be relatively close to the center of the window and can be reduced in the windowed correlation curve 1306 as illustrated by corresponding peak 1316. The windowing operation can reduce the number of peaks to consider and reduce the influence of distant peaks on the heart rate prediction algorithm.

Although discussed above as separate heuristics, some exemplary heuristics can operate in concert with other heuristics in some implementations or can be omitted from other implementations.

In some examples, a low confidence measure can be used to adjust properties of the heart rate determination system. For example, a low confidence measure or a threshold number of low confidence measures can cause the system to adjust the window generator 206 parameters (e.g., smaller or larger window, or a different windowing function), the sampling frequency of sensors, or the DFT circuitry in order to improve performance. In other examples, a low confidence measure or a threshold number of low confidence measures can be used to provide a message to a user that heart rate cannot be determined with a high degree of confidence, provide a message to the user to better secure the device implementing the system to the skin, or activate additional compensation, among other possible functions.

Referring back to FIG. 2, the output unit 260 can receive predicted heart rate values and confidence measures from the HTC unit 250. The output unit 260 can include a memory or buffer. As discussed above, in some cases the output unit can store a history of heart rate prediction and confidence measure values in the memory. The output can include a heart rate prediction and a confidence measure that can be suitable for additional processing or transmission to the I/O unit 110 of FIG. 1.

The acceleration removal algorithm and HTC algorithms can provide a variety of advantages in addition to improved accuracy in removing motion artifacts and predicting a user's heart rate. For example, the HTC algorithm can provide for conceptual simplicity in predicting a heart rate based on comparison with templates from a library. Additionally, operating in the frequency domain can provide advantages by concentrating periodic heart rate information distributed over length of time into narrow regions (i.e., peaks). Comparing and correlating time domain templates with time domain signals can be computationally expensive and require storing significant amounts of data. In contrast, frequency domain spectral templates can be represented with minimal information. Additionally, the HTC algorithm provides scale invariance, i.e., ignores the scaling differences between the time domain PPG and acceleration signals, and robustness against signal misalignment, i.e., due to physiological delays between acceleration and its representation in PPG signals, by focusing on removing the magnitude of the acceleration signal represented in the PPG signal in the frequency domain. As discussed above, heuristics such as the cone of reachability can also provide immunity to short term motion artifacts. Finally, the algorithm can leverage FFT algorithms readily available in existing chips for efficient implementation.

FIG. 14 illustrates an exemplary algorithm for removing acceleration artifacts from a PPG signal according to examples of the disclosure. As discussed above, in some examples, a heart rate prediction system can remove motion artifacts from the PPG signals before applying the HTC algorithm. The system can generate windowed PPG and acceleration signals (1400). As discussed above, in some examples, the window can include 8 seconds of PPG and acceleration data. The windowed signals can be generated using a window generator function. The system can convert the windowed PPG and acceleration signals into the frequency domain (1405). In the frequency domain, the PPG and acceleration signals can be represented by complex coefficients including magnitude and phase. In some examples the phase component can be ignored. The system can generate an acceleration mask (1410). The acceleration mask can be generated using the magnitude portion of the frequency domain representation of the acceleration signals. A mean of the magnitudes of the acceleration signal can be calculated. In some examples, the acceleration mask can be computed to be the maximum of the inverse of the magnitude of the acceleration signal coefficients or unity for all magnitudes greater than the mean (and unity for all magnitudes less than the mean). The acceleration mask can also truncate the number of coefficients used to the range of frequencies of interest (e.g., less than 20 Hz). The system can also adjust the magnitude of and truncate the PPG signals (1415). In other words, the frequency domain representation of the PPG signals can be limited to the magnitude representation of the coefficients and to the frequency range of interest (e.g., less than 20 Hz). The system can filter acceleration (motion artifacts) from the PPG signals using the acceleration mask (1420). In some examples, the acceleration mask can operate as a matched filter matched to the frequencies corresponding to the acceleration contributions. The portions of the acceleration mask with a value of unity can leave the truncated PPG signals unchanged, but the portions of the acceleration masks with inverted peaks can reduce or completely remove the corresponding peaks in the frequency domain PPG signal corresponding to the acceleration.

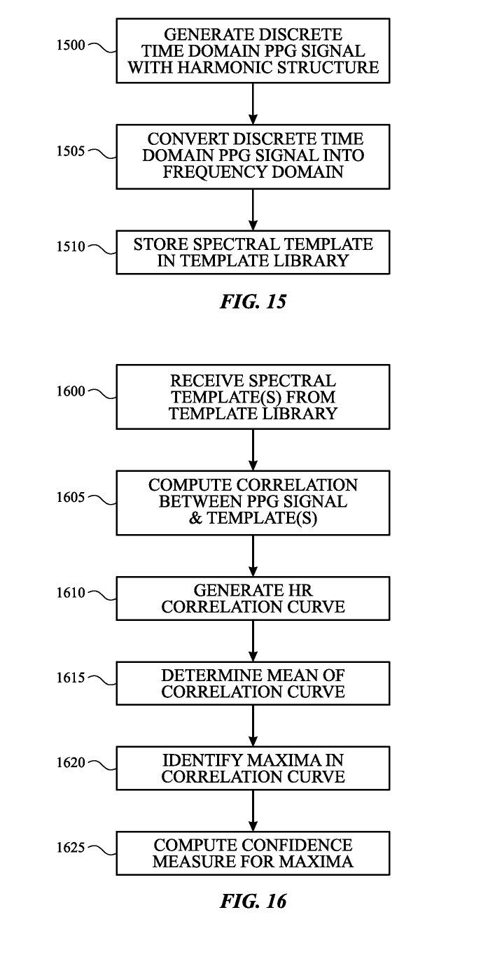

FIG. 15 illustrates an exemplary algorithm for generating and storing spectral templates according to examples of the disclosure. The system can generate discrete time domain heart rate signals with harmonic structure (1500). For each candidate heart rate, a discrete time domain template signal can be generated by a synthetic waveform generator. The discrete time domain template signal can have a composite sinusoidal structure made up of separate sinusoidal components (e.g. 4 components). The function generated using equation (2) can be represented more compactly in the frequency domain (e.g., using 8 real numbers along with a frequency parameter) and can have unique spectral line signatures. The system can convert the discrete time domain template signals into the frequency domain (1505). For example, an FFT can be used to generate a spectral template including magnitude coefficients (i.e., a frequency domain representation of the time domain template signals). In some examples, the spectral template can include four harmonics. The number of coefficients in the spectral template can be limited to a frequency range of interest (e.g., less than 20 Hz). The system can store the spectral template in a template library (1510). In some examples, the templates can be generated externally to the system and the spectral templates stored in the system for implementing the HTC algorithm.

FIG. 16 illustrates an exemplary algorithm for generating a heart rate correlation curve and confidence measure according to examples of the disclosure. A correlation unit can receive one or more spectral templates from the template library (1600). As discussed above, the spectral templates can correspond to various heart rates, and in some examples a spectral template can be generated for each integer heart rate in a range of possible heart rates (e.g., 35 bpm to 220 bpm). The system can compute a correlation between the PPG signals and the one or more templates (1605). In some examples, a correlation can be computed between the PPG signals and each spectral template in a range of possible heart rates. The system can generate a heart rate correlation curve (1610). The correlation curve can represent the correlation value associated with a corresponding spectral template frequency. The system can determine a mean of the covariance curve (1615). The system can also identify one or more maxima in the correlation curve (1620). The system can compute a confidence measure for the one or more maxima (1625). In some examples, the confidence measure can be based on the mean and the correlation value at the one or more maxima. For example, the confidence measure can be the ratio of the correlation value at a maximum to the mean, where a high value of the ratio can indicate high confidence that the corresponding frequency represents the true heart rate. Other confidence measurements can be computed as discussed above.

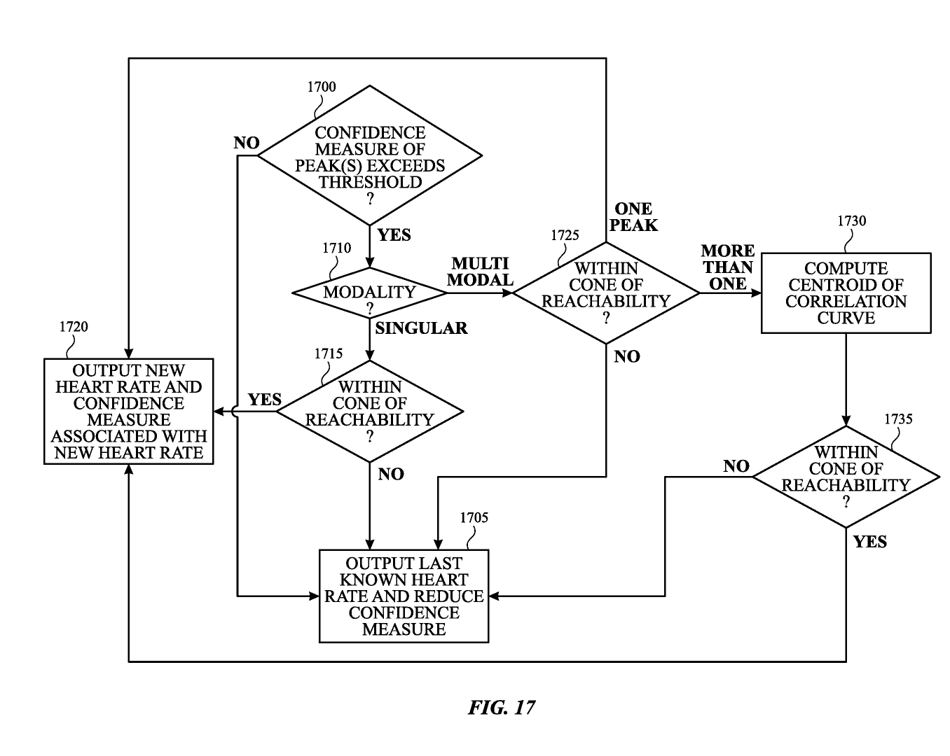

FIG. 17 illustrates exemplary heuristics for determining a predicted heart rate according to examples of the disclosure. The heuristics unit can receive information including a correlation curve, one or more candidate heart rate predictions, and/or one or more corresponding confidence measures. The system can determine whether the one or more confidence measures corresponding to the one or more peaks exceeding a threshold (1700). If none of the peaks exceeds the threshold, the output heart rate prediction of the heuristics unit can be can be the last known heart rate and the confidence measure associated with the output heart rate prediction can be reduced from the confidence measure associated with the last known heart rate (1705). If one or more of the peaks exceeds the threshold, the system can determine the modality of the correlation curve (1710). If the system determines a singular mode (i.e., one peak exceeding the confidence measure threshold) in the correlation curve, the system can determine if the peak is within a cone of reachability (1715). If the singular mode peak is within the cone of reachability, the system can output the new heart rate prediction and the confidence measure associated with the new heart rate prediction (1720). If the singular mode peak is not within the cone of reachability, the output heart rate can be the last known heart rate and the confidence measure associated with the output heart rate can be reduced from the confidence measure associated with the last known heart rate (1705). If the system determines a multimodal correlation curve, the system can determine whether the peaks in the correlation curve are within the cone of reachability (1725). If none of the peaks is within the cone of reachability, the output heart rate can be the last known heart rate and the confidence measure associated with the output heart rate can be reduced from the confidence measure associated with the last known heart rate (1705). If one of the peaks is within the cone of reachability, the system can output the new heart rate prediction and the confidence measure associated with the new heart rate prediction (1720). If more than one of the peaks is within the cone of reachability, the system can compute a centroid of the correlation curve (1730). The centroid can be weighted according to the correlation values at each frequency. The system can determine whether the frequency centroid is within the cone of reachability (1735). If the frequency centroid is not within the cone of reachability, the output heart rate can be the last known heart rate and the confidence measure associated with the output heart rate can be reduced from the confidence measure associated with the last known heart rate (1705). If the centroid is within the cone of reachability, the system can output the new heart rate prediction and the confidence measure associated with the new heart rate prediction (1720). Although FIG. 17 illustrates an example operation of the heuristics unit, other heuristics and various combinations of heuristics can be used to predict heart rate.

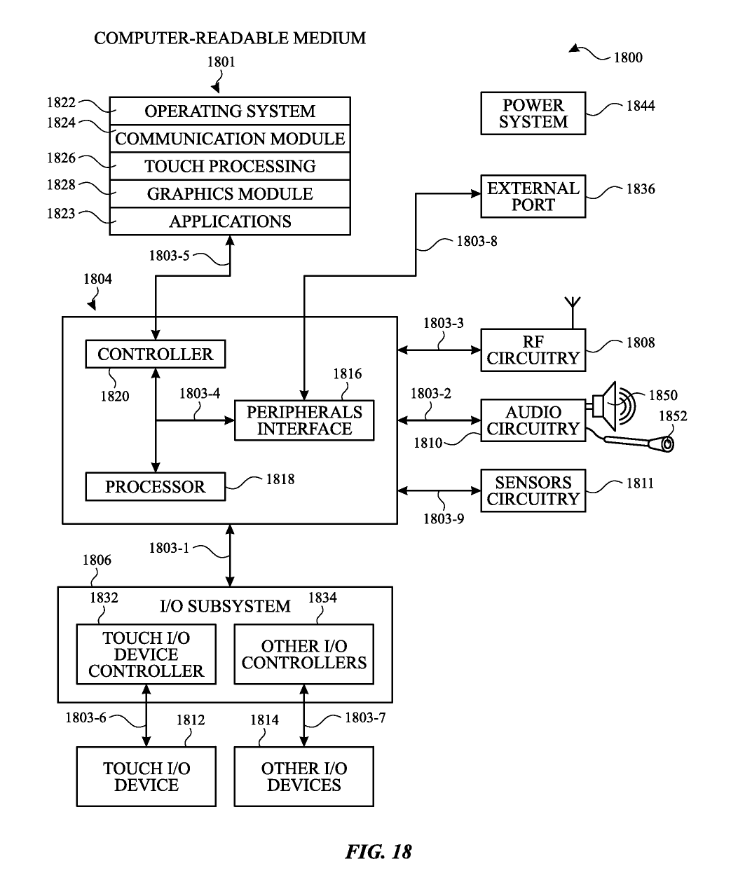

A system architecture implementing the acceleration removal and HTC algorithms can be included in any portable or non-portable device including but not limited to a wearable device (e.g., smart band, health band, smart watch), a communication device (e.g., mobile phone, smart phone), a multi-media device (e.g., MP3 player, TV, radio), a portable or handheld computer (e.g., tablet, netbook, laptop), a desktop computer, an All-In-One desktop, a peripheral device, or any other system or device adaptable to the inclusion of system architecture, including combinations of two or more of these types of devices. FIG. 18 illustrates a block diagram of an exemplary system architecture 1800 that can implement the acceleration removal and/or HTC algorithms according to examples of the disclosure. System architecture 1800 can generally include one or more computer-readable media 1801, processing system 1804, I/O subsystem 1806, radio frequency (RF) circuitry 1808, audio circuitry 1810, and sensors circuitry 1811. These components can be coupled by one or more communication buses or signal lines 1803.

It should be understood that the exemplary architecture shown in FIG. 18 can have more or fewer components than shown, or a different configuration of components. The various components shown in FIG. 18 can be implemented in hardware, software, firmware or any combination thereof, including one or more signal processing and/or application specific integrated circuits.

RF circuitry 1808 can be used to send and receive information over a wireless link or network to one or more other devices and includes well-known circuitry for performing this function. RF circuitry 1808 and audio circuitry 1810 can be coupled to processing system 1804 via peripherals interface 1816. Peripherals interface 1816 can include various known components for establishing and maintaining communication between peripherals and processing system 1804. Audio circuitry 1810 can be coupled to audio speaker 1850 and microphone 1852 and can include known circuitry for processing voice signals received from peripherals interface 1816 to enable a user to communicate in real-time with other users. In some examples, audio circuitry 1810 can include a headphone jack (not shown). Sensors circuitry 1811 can be coupled to various sensors including, but not limited to, one or more light emitting diodes (LEDs) or other light emitters, one or more photodiodes or other light sensors, one or more photothermal sensors, a magnetometer, an accelerometer, a gyroscope, a barometer, a compass, a proximity sensor, a camera, an ambient light sensor, a thermometer, a global positioning system (GPS) sensor, and various system sensors which can sense remaining battery life, power consumption, processor speed, CPU load, and the like.

Peripherals interface 1816 can couple the input and output peripherals of the system 1800 to one or more processors 1818 and one or more computer-readable mediums 1801 via a controller 1820. The one or more processors 1818 communicate with the one or more computer-readable media 1801 via the controller 1820. The one more computer-readable media 1801 can be any device or medium that can store code and/or data for use by the one or more processors 1818. In some examples, medium 1801 can be a non-transitory computer-readable storage medium. Medium 1801 can include a memory hierarchy, including but not limited to cache, main memory and secondary memory. The memory hierarchy can, as non-limiting examples, be implemented using any combination of RAM (e.g., SRAM, DRAM, DDRAM), ROM, FLASH, magnetic and/or optical storage devices, such as disk drives, magnetic tape, compact disks (CDs) and digital video discs (DVDs). Medium 1801 can also include a transmission medium for carrying information-bearing signals indicative of computer instructions or data (with or without a carrier wave upon which the signals can be modulated). For example, the transmission medium can include a communications network, including but not limited to the Internet (also referred to as the World Wide Web), intranet(s), Local Area Networks (LANs), Wide Local Area Networks (WLANs), Storage Area Networks (SANs), Metropolitan Area Networks (MAN) and the like.

One or more processors 1818 can run various software components stored in medium 1801 to perform various functions for system architecture 1800. In some examples, the software components can include operating system 1822, communication module (or set of instructions) 1824, touch processing module (or set of instructions) 1826, graphics module (or set of instructions) 1828, and one or more applications (or set of instructions) 1823. Each of these modules and above noted applications can correspond to a set of instructions for performing one or more functions described above and the methods described in this application (e.g., the computer-implemented methods and other information processing methods described herein). These modules (i.e., sets of instructions) need not be implemented as separate software programs, procedures or modules, and thus various subsets of these modules can be combined or otherwise re-arranged in various examples. In some examples, medium 1801 can store a subset of the modules and data structures identified above. Furthermore, medium 1801 can store additional modules and data structures not described above.

Operating system 1822 can include various procedures, sets of instructions, software components and/or drivers for controlling and managing general system tasks (e.g., memory management, storage device control, power management, etc.) and facilitates communication between various hardware and software components.

Communication module 1824 can facilitate communication with other devices over one or more external ports 1836 or via RF circuitry 1808 and can include various software components for handling data received from RF circuitry 1808 and/or external port 1836.

Graphics module 1828 can include various known software components for rendering, animating and displaying graphical objects on a display surface. In examples in which touch I/O device 1812 is a touch sensing display (e.g., touch screen), graphics module 1828 can include components for rendering, displaying, and animating objects on the touch sensing display. The touch I/O device 1812 and/or the other I/O device 1814 can comprise the I/O unit 110 of FIG. 1, and can also incorporate a UI interface permitting a use to select among programming modes of displaying heart rate data when the I/O device is incorporated into a device 112 of FIG. 1. Further, in relation to FIG. 1, the light emitter 102 and light sensor 104 can be part of the I/O device 1814, and the touch screen 120 can correspond to the touch I/O device 1812 of FIG. 18. The I/O unit 110 either integrated within device 112 or via coupling to microphone/speaker 122 can also provide audio outputs as part of the user communications corresponding to audio circuitry 1810 of FIG. 18. Microphone 1852 of FIG. 18 can correspond to the microphone/speaker 122 of FIG. 1.

One or more applications 1823 can include any applications installed on system 1800, including without limitation, a browser, address book, contact list, email, instant messaging, word processing, keyboard emulation, widgets, JAVA-enabled applications, encryption, digital rights management, voice recognition, voice replication, location determination capability (such as that provided by the GPS), a music player, etc.

Touch processing module 1826 can include various software components for performing various tasks associated with touch I/O device 1812 including but not limited to receiving and processing touch input received from touch I/O device 1812 via touch I/O device controller 1832.

I/O subsystem 1806 can be coupled to touch I/O device 1812 and one or more other I/O devices 1814 for controlling or performing various functions. Touch I/O device 1812 can communicate with processing system 1804 via touch I/O device controller 1832, which can include various components for processing user touch input (e.g., scanning hardware). One or more other input controllers 1834 can receive/send electrical signals from/to other I/O devices 1814. Other I/O devices 1814 can include physical buttons, dials, slider switches, sticks, keyboards, touch pads, additional display screens, or any combination thereof.

If embodied as a touch screen, touch I/O device 1812 can display visual output to the user in a GUI. The visual output can include text, graphics, video, and any combination thereof. Some or all of the visual output can correspond to user-interface objects. Touch I/O device 1812 can form a touch sensing surface that accepts touch input from the user. Touch I/O device 1812 and touch screen controller 1832 (along with any associated modules and/or sets of instructions in medium 1801) can detect and track touches or near touches (and any movement or release of the touch) on touch I/O device 1812 and can convert the detected touch input into interaction with graphical objects, such as one or more user-interface objects. In the case in which touch I/O device 1812 is embodied as a touch screen, the user can directly interact with graphical objects that can be displayed on the touch screen. Alternatively, in the case in which touch I/O device 1812 is embodied as a touch device other than a touch screen (e.g., a touch pad), the user can indirectly interact with graphical objects that can be displayed on a separate display screen embodied as I/O device 1814.

Touch I/O device 1812 can be analogous to the multi-touch sensing surface described in the following U.S. Pat. No. 6,323,846 (Westerman et al.), U.S. Pat. No. 6,570,557 (Westerman et al.), and/or U.S. Pat. No. 6,677,932 (Westerman), and/or U.S. Patent Publication 2002/0015024A1.

In examples for which touch I/O device 1812 is a touch screen, the touch screen can use liquid crystal display (LCD) technology, light emitting polymer display (LPD) technology, organic LED (OLED), or organic electro luminescence (OEL), although other display technologies can be used in other examples.

Feedback can be provided by touch I/O device 1812 based on the user's touch input as well as a state or states of what is being displayed and/or of the computing system. Feedback can be transmitted optically (e.g., light signal or displayed image), mechanically (e.g., haptic feedback, touch feedback, force feedback, or the like), electrically (e.g., electrical stimulation), olfactory, acoustically (e.g., beep or the like), or the like or any combination thereof and in a variable or non-variable manner.

System architecture 1800 can also include power system 1844 for powering the various hardware components and can include a power management system, one or more power sources, a recharging system, a power failure detection circuit, a power converter or inverter, a power status indicator and any other components typically associated with the generation, management and distribution of power in portable devices.

In some examples, peripherals interface 1816, one or more processors 1818, and memory controller 1820 of the processing system 1804 can be implemented on a single chip. In some other examples, they can be implemented on separate chips.

Therefore, according to the above, some examples of the disclosure are directed to a device for determining a heart rate. The device can comprise a sensor configured to generate first signals and processing circuitry capable of: generating a frequency domain representation of the first signals; correlating the frequency domain representation of the first signals and a plurality of spectral heart rate templates to generate a plurality of correlation values; and predicting a heart rate based on the plurality of correlation values. Additionally or alternatively to one or more of the examples disclosed above, the device can further comprise a memory coupled to the processing circuitry and configured to store the plurality of spectral heart rate templates. The processing circuitry can further capable of accessing the plurality of spectral heart rate templates stored in the memory. Additionally or alternatively to one or more of the examples disclosed above, the processing circuitry can further capable of identifying one or more maxima in the plurality of correlation values. The one or more frequencies associated with the one or more maxima can be candidates for predicting the heart rate. Additionally or alternatively to one or more of the examples disclosed above, the processing circuitry can be further capable of generating one or more confidence measures, the one or more confidence measures corresponding to the one or more maxima. Additionally or alternatively to one or more of the examples disclosed above, generating a confidence measure can include calculating a mean of the correlation values and calculating a ratio of a correlation value at a maxima to the mean of the correlation values. Additionally or alternatively to one or more of the examples disclosed above, the processing circuitry can be further capable of applying one or more heuristics for predicting the heart rate based on the correlation values and the one or more confidence measures. Additionally or alternatively to one or more of the examples disclosed above, the device further comprising: a sensor configured to generate acceleration signals. The processor can be further capable of: generating a frequency domain representation of the acceleration signals; and removing motion artifacts from the frequency domain representation of the first signals using the frequency domain representation of the acceleration signals. Additionally or alternatively to one or more of the examples disclosed above, removing the motion artifacts can further include truncating the frequency domain representations of the acceleration signals and first signals to exclude information outside a frequency range of interest. Additionally or alternatively to one or more of the examples disclosed above, the processor can be further capable of: determining a mean of the frequency domain representation of the acceleration signals; generating acceleration mask signals based on the frequency domain representation of the acceleration signals and the mean of the frequency domain representation of the acceleration signals; and filtering the frequency domain representation of the first signals using the acceleration mask signals to remove the motion artifacts.

Some examples of the disclosure are directed to a method executed by processing circuitry for predicting a heart rate. The method can comprise: receiving first signals generated by a sensor; generating a frequency domain representation of the first signals; correlating the frequency domain representation of the first signals and a plurality of spectral heart rate templates to generate a plurality of correlation values; and predicting a heart rate based on the plurality of correlation values. Additionally or alternatively to one or more of the examples disclosed above, the method can further comprise generating a spectral heart rate template for a given heart rate by synthesizing a composite time domain waveform including a plurality of periodic components at one or more frequencies corresponding to the given heart rate and transforming the composite time domain waveform into the frequency domain. Additionally or alternatively to one or more of the examples disclosed above, the method can further comprise determining a number of coefficients for the spectral heart rate template for a given heart rate by the number of coefficients that occur within a frequency range of interest. Additionally or alternatively to one or more of the examples disclosed above, the method can further comprise identifying one or more maxima in the plurality of correlation values. The one or more frequencies associated with the one or more maxima can be candidates for predicting the heart rate. Additionally or alternatively to one or more of the examples disclosed above, the method can further comprise generating one or more confidence measures. The one or more confidence measures can correspond to the one or more maxima. Additionally or alternatively to one or more of the examples disclosed above, the method can further comprise calculating a mean of the correlation values; and generating one or more confidence measures based on a ratio of a correlation value at one or more maxima to the mean of the correlation values. Additionally or alternatively to one or more of the examples disclosed above, the method can further comprise applying one or more heuristics for predicting the heart rate based on the correlation values and the one or more confidence measures. Additionally or alternatively to one or more of the examples disclosed above, the one or more heuristics can include comparing a confidence measure to a confidence measure threshold. Additionally or alternatively to one or more of the examples disclosed above, the one or more heuristics can include comparing a frequency associated with a maximum in the correlation values with one or more recent heart rate predictions. Additionally or alternatively to one or more of the examples disclosed above, the one or more heuristics can include calculating a centroid of the correlation values. Additionally or alternatively to one or more of the examples disclosed above, the one or more heuristics can include determining a modality of the correlation values. Additionally or alternatively to one or more of the examples disclosed above, the one or more heuristics can include windowing the correlation values around a last known heart rate prediction. Additionally or alternatively to one or more of the examples disclosed above, the method can further comprise receiving acceleration signals from an accelerometer; generating a frequency domain representation of the acceleration signals; and removing motion artifacts from the frequency domain representation of the first signals using the frequency domain representation of the acceleration signals. Additionally or alternatively to one or more of the examples disclosed above, the method can further comprise: truncating the frequency domain representations of the acceleration signals and first signals to exclude information outside a frequency range of interest. Additionally or alternatively to one or more of the examples disclosed above, the method can further comprise: determining a mean of the frequency domain representation of the acceleration signals; generating acceleration mask signals based on the frequency domain representation of the acceleration signals and the mean of the frequency domain representation of the acceleration signals; and filtering the frequency domain representation of the first signals using the acceleration mask signals to remove the motion artifacts.

Some examples of the disclosure are directed to non-transitory computer readable storage medium. The computer readable medium can contain instructions that, when executed, perform a method for operating an electronic device. The electronic device can include a processor. The method can comprise: receiving first signals generated by a sensor; generating a frequency domain representation of the first signals; correlating the frequency domain representation of the first signals and a plurality of spectral heart rate templates to generate a plurality of correlation values; and predicting a heart rate based on the plurality of correlation values. Additionally or alternatively to one or more of the examples disclosed above, the method can further comprise generating a spectral heart rate template for a given heart rate by synthesizing a composite time domain waveform including a plurality of periodic components at one or more frequencies corresponding to the given heart rate and transforming the composite time domain waveform into the frequency domain. Additionally or alternatively to one or more of the examples disclosed above, the method can further comprise determining a number of coefficients for the spectral heart rate template for a given heart rate by the number of coefficients that occur within a frequency range of interest. Additionally or alternatively to one or more of the examples disclosed above, the method can further comprise identifying one or more maxima in the plurality of correlation values. The one or more frequencies associated with the one or more maxima can be candidates for predicting the heart rate. Additionally or alternatively to one or more of the examples disclosed above, the method can further comprise generating one or more confidence measures. The one or more confidence measures can correspond to the one or more maxima. Additionally or alternatively to one or more of the examples disclosed above, the method can further comprise calculating a mean of the correlation values; and generating one or more confidence measures based on a ratio of a correlation value at one or more maxima to the mean of the correlation values. Additionally or alternatively to one or more of the examples disclosed above, the method can further comprise applying one or more heuristics for predicting the heart rate based on the correlation values and the one or more confidence measures. Additionally or alternatively to one or more of the examples disclosed above, the one or more heuristics can include comparing a confidence measure to a confidence measure threshold. Additionally or alternatively to one or more of the examples disclosed above, the one or more heuristics can include comparing a frequency associated with a maximum in the correlation values with one or more recent heart rate predictions. Additionally or alternatively to one or more of the examples disclosed above, the one or more heuristics can include calculating a centroid of the correlation values. Additionally or alternatively to one or more of the examples disclosed above, the one or more heuristics can include determining a modality of the correlation values. Additionally or alternatively to one or more of the examples disclosed above, the one or more heuristics can include windowing the correlation values around a last known heart rate prediction. Additionally or alternatively to one or more of the examples disclosed above, the method can further comprise receiving acceleration signals from an accelerometer; generating a frequency domain representation of the acceleration signals; and removing motion artifacts from the frequency domain representation of the first signals using the frequency domain representation of the acceleration signals. Additionally or alternatively to one or more of the examples disclosed above, the method can further comprise: truncating the frequency domain representations of the acceleration signals and first signals to exclude information outside a frequency range of interest. Additionally or alternatively to one or more of the examples disclosed above, the method can further comprise: determining a mean of the frequency domain representation of the acceleration signals; generating acceleration mask signals based on the frequency domain representation of the acceleration signals and the mean of the frequency domain representation of the acceleration signals; and filtering the frequency domain representation of the first signals using the acceleration mask signals to remove the motion artifacts.

Although the disclosed examples have been fully described with reference to the accompanying drawings, it is to be noted that various changes and modifications will become apparent to those skilled in the art. Such changes and modifications are to be understood as being included within the scope of the disclosed examples as defined by the appended claims.

What is claimed as new and desired to be protected by Letters Patent of the United States is:

1. A device for predicting heart rate, comprising:
a sensor configured to generate first signals; and
processing circuitry capable of:
generating a frequency domain representation of the first signals;
correlating the frequency domain representation of the first signals and a plurality of spectral heart rate templates to generate a plurality of correlation values; and
predicting a heart rate based on the plurality of correlation values.

2. The device of claim 1, further comprising:
a memory coupled to the processing circuitry and configured to store the plurality of spectral heart rate templates, wherein the processing circuitry is further capable of accessing the plurality of spectral heart rate templates stored in the memory.

3. The device of claim 1, wherein the processing circuitry is further capable of identifying one or more maxima in the plurality of correlation values, wherein one or more frequencies associated with the one or more maxima are candidates for predicting the heart rate.

4. The device of claim 3, wherein the processing circuitry is further capable of generating one or more confidence measures, the one or more confidence measures corresponding to the one or more maxima.

5. The device of claim 4, wherein generating a confidence measure includes calculating a mean of the correlation values and calculating a ratio of a correlation value at a maxima to the mean of the correlation values.

6. The device of claim 4, wherein the processing circuitry is further capable of applying one or more heuristics for predicting the heart rate based on the correlation values and the one or more confidence measures.

7. The device of claim 1, further comprising: a sensor configured to generate acceleration signals; wherein the processor is further capable of:
generating a frequency domain representation of the acceleration signals; and
removing motion artifacts from the frequency domain representation of the first signals using the frequency domain representation of the acceleration signals.

8. The device of claim 7, wherein removing the motion artifacts further includes truncating the frequency domain representations of the acceleration signals and first signals to exclude information outside a frequency range of interest.

9. The device of claim 7, wherein the processor is further capable of:
determining a mean of the frequency domain representation of the acceleration signals;
generating acceleration mask signals based on the frequency domain representation of the acceleration signals and the mean of the frequency domain representation of the acceleration signals; and
filtering the frequency domain representation of the first signals using the acceleration mask signals to remove the motion artifacts.

10. A method executed by processing circuitry for predicting a heart rate, the method comprising:
receiving first signals generated by a sensor;
generating a frequency domain representation of the first signals;
correlating the frequency domain representation of the first signals and a plurality of spectral heart rate templates to generate a plurality of correlation values; and
predicting a heart rate based on the plurality of correlation values.

11. The method of claim 10, further comprising generating a spectral heart rate template for a given heart rate by synthesizing a composite time domain waveform including a plurality of periodic components at one or more frequencies corresponding to the given heart rate and transforming the composite time domain waveform into the frequency domain.

12. The method of claim 11, further comprising determining a number of coefficients for the spectral heart rate template for a given heart rate by the number of coefficients that occur within a frequency range of interest.

13. The method of claim 10, further comprising identifying one or more maxima in the plurality of correlation values, wherein one or more frequencies associated with the one or more maxima are candidates for predicting the heart rate.

14. The method of claim 13, further comprising generating one or more confidence measures, the one or more confidence measures corresponding to the one or more maxima.

15. The method of claim 14, further comprising:
calculating a mean of the correlation values; and
generating one or more confidence measures based on a ratio of a correlation value at one or more maxima to the mean of the correlation values.

16. The method of claim 14, further comprising applying one or more heuristics for predicting the heart rate based on the correlation values and the one or more confidence measures.

17. The method of claim 16, wherein the one or more heuristics includes comparing a confidence measure to a confidence measure threshold.

18. The method of claim 16, wherein the one or more heuristics includes comparing a frequency associated with a maximum in the correlation values with one or more recent heart rate predictions.

19. The method of claim 16, wherein the one or more heuristics includes calculating a centroid of the correlation values.

20. The method of claim 16, wherein the one or more heuristics includes determining a modality of the correlation values.

21. The method of claim 16, wherein the one or more heuristics includes windowing the correlation values around a last known heart rate prediction.

22. The method of claim 10, further comprising:
receiving acceleration signals from an accelerometer;
generating a frequency domain representation of the acceleration signals; and
removing motion artifacts from the frequency domain representation of the first signals using the frequency domain representation of the acceleration signals.

23. The method of claim 22, further comprising:
truncating the frequency domain representations of the acceleration signals and first signals to exclude information outside a frequency range of interest.

24. The method of claim 22, further comprising:
determining a mean of the frequency domain representation of the acceleration signals;
generating acceleration mask signals based on the frequency domain representation of the acceleration signals and the mean of the frequency domain representation of the acceleration signals; and
filtering the frequency domain representation of the first signals using the acceleration mask signals to remove the motion artifacts.

25. A non-transitory computer readable storage medium, the computer readable medium containing instructions that, when executed, perform a method for operating an electronic device, the electronic device including a processor, the method comprising:
receiving first signals generated by a sensor when positioned on or adjacent to a user's skin;
generating a frequency domain representation of the first signals;
correlating the frequency domain representation of the first signals and a plurality of spectral heart rate templates to generate a plurality of correlation values; and
predicting a heart rate of user based on the plurality of correlation values.
